SEQUENCE LISTING (4 sequences)

SEQ ID NO: 1 - DNA, 20 bp, Rattus norvegicus
ggctacagcttcaccaccac

SEQ ID NO: 2 - DNA, 16 bp, Rattus norvegicus
tgcgctcaggaggagc

SEQ ID NO: 3 - DNA, 21 bp, Rattus norvegicus
aggcaagagggagttaccact

SEQ ID NO: 4 - DNA, 16 bp, Rattus norvegicus
ggtggccaaggggatg

(19) United States
(12) Patent Application Publication
(10) Pub. No.: US 2006/0177853 A1
(43) Pub. Date: Aug. 10, 2006

(54) Antidepressant drug target

(75) Inventors: Tamas Bartfai, La Jolla, CA (US); Bruno Conti, San Diego, CA (US); Xiaoying Lu, La Jolla, CA (US)

Correspondence Address: SCHWEGMAN, LUNDBERG, WOESSNER & KLUTH, P.A., P.O. BOX 2938, MINNEAPOLIS, MN, 55402, US

(73) Assignee: The Scripps Research Institute

(21) Appl. No.: 11/324,770

(22) Filed: Jan. 3, 2006

Related U.S. Application Data
(60) Provisional application No. 60/641,052, filed on Jan. 3, 2005.

(51) Int. Cl. C12Q 1/68

(52) U.S. Cl. 435/6

(57) ABSTRACT

The galaninergic system of the dorsal raphe nucleus is employed as a target for screening drug candidates for antidepressant activity.

### CROSS-REFERENCE TO RELATED APPLICATIONS

This application claims the benefit of the filing date of U.S. application Ser. No. 60/641,052, filed on Jan. 3, 2005, the disclosure of which is incorporated by reference herein.

### FIELD OF INVENTION

The invention relates to a target for screening antidepressant drug candidates. More specifically, the invention relates to processes that assay the upregulation of the galaninergic system of the dorsal raphe nucleus for screening antidepressant drug candidates.

### BACKGROUND

The molecular mechanism of action of fluoxetine (FLX), beyond its effect of elevating synaptic serotonin (5-HT) concentration, is not fully understood. The delay in the onset of clinical antidepressant effect suggests that transcriptional and translational events, leading to functional changes in signaling within the major serotoninergic nucleus dorsal raphe nucleus (DRN) and in its projection areas, may be required for these therapeutic effects (Duman, R. S., et al. Arch. Gen. Psychiatry 1997, 54, 597-606; Mann, J. J. Neuropsychopharmacology 1999, 21, 99S-105S; and Schloss, P., et al. Pharmacol. Ther. 2004, 102, 47-60). One potential player in mediating the long term effects of FLX, besides 5-HT, is the neuropeptide galanin. Galanin, through its three G protein coupled receptors, GalR1, GalR2 and GalR3 (Branchek, T. A., et al. Trends Pharmacol. Sci. 2000, 21, 109-17), regulates homeostatic and motivated behaviors including pain perception, sleep, food intake, sexual activity and learning and memory (Vrontakis, M. E. Curr. Drug Targets CNS Neurol. Disord. 2002, 1, 53141). Galaninergic transmission modulates the activity of monoaminergic neurons in the ventral tegmental area, DRN and locus coeruleus (LC) (Wrenn, C. C., et al. Prog. Neuropsychopharmacol. Biol. Psychiatry 2001, 25, 283 99; Xu, Z. Q., et al. Neuroscience 1998, 87, 79 94; Weiss, J. M., et al. Ann. N.Y. Acad. Sci. 1998, 863, 364-82; Melander, T., et al. Journal of Neuroscience 1986, 6, 3640-54; and Skofitsch, G., et al. Peptides 1985, 6, 509-46). Galanin receptor subtypes GalR1 (Xu, Z. Q., et al. Neuroscience 1998, 87, 79-94) and GalR2 are expressed in DRN neurons (O'Donnell, D., et al. J. Comp. Neurol. 1999, 409, 469-81) that can be activated by galanin dendritically released from the dorsal raphe 5-HT neurons (Melander, T., et al. Journal of Neuroscience 1986, 6, 3640-54; and Priestley, J. V., et al. J. Neurosci. Methods 1993, 48, 99-110) or from surrounding galanin immunoreactive terminals (Xu, Z. Q., et al. Neuroscience 1998, 87, 79-94). In the noradrenergic nucleus LC, an area that is closely connected both structurally and functionally to DRN (Gallager, D. W., et al., Neuropharmacology, 1976, 15, 149-56; and Aghajanian, G. K., et al. Brain Res. 1978, 153, 169-75), GalR1 expression is induced by morphine withdrawal (Zachariou, V., et al. Journal of Neurochemistry 2001, 76, 191-200), and the galanin receptor agonist, galnon, was shown to attenuate several withdrawal signs (Zachariou, V., et al. Proc. Natl. Acad. Sci. U.S.A. 2003, 100, 9028-33).

It is worth noting that drug withdrawal often precipitates symptoms of depression, and depression is a commonly observed withdrawal symptom in humans (Barr, A. M., et al. Trends Pharmacol. Sci. 2002, 23, 475-82; and Markou, A., et al. Neuropsychopharmacology 1991, 4, 17-26). In addition, decreased galanin expression in DRN, hippocampus and hypothalamus have been observed in rat models of depression (Bellido, I., et al. Neuroscience Letters 2002, 317, 101-5; Husum, H., et al. Neuropsychopharmacology 2003, 28, 1292-9; and Sergeyev, V., et al. Psychopharmacology 2005, 178, 115-124). A recent clinical study reported preliminary evidence for an acute antidepressant effect of galanin (i.v.) in depressed patients (Murck, H., et al. Psychoneuroendocrinology 2004, 29, 1205-11), while a few early microdialysis and behavioral studies in rodents suggested depressive actions of galanin (Wrenn, C. C., et al. Prog. Neuropsychopharmacol. Biol. Psychiatry 2001, 25, 283-99; Weiss, J. M., et al. Ann. N.Y. Acad. Sci. 1998, 863, 364-82; Yoshitake, T., et al. Neuropharmacology 2003, 44, 206-13; and Yoshitake, T., et al. Proc. Natl. Acad. Sci. U.S.A. 2004, 101, 354-9). It has earlier been observed that a systemically active galanin receptor agonist, galmic, in a dose that suppresses status epilepticus, has an antidepressant like effect in the forced swim test (Bartfai, T., et al. Proc. Natl. Acad. Sci. U.S.A. 2004, 101, 10470-5).

Thus, there is a need for methods to identify agents with specific antidepressant activity.

### SUMMARY OF THE INVENTION

According to the invention galaninergic transmission in the DRN and LC is enhanced by several different antidepressant treatments within time frames that are relevant to their therapeutic effects. In addition, a galanin receptor antagonist, M40, blocked the antidepressant like effect of FLX in the rat forced swim test, suggesting that the galaninergic system contributes to the antidepressant like effect of FLX. Moreover, a galanin receptor agonist produced an antidepressant like effect in the same behavioral test. The combined data shows that the galaninergic system is a target for antidepressant therapies.

Thus, the invention provides a method for screening for a test agent with antidepressant activity. The method includes treating a subject with a therapeutic dose of the test agent. The subject may be a human or non-human animal, including an avian, bovine, equine, ovine, caprine, swine, feline, canine or rodent, e.g., mouse, rat, rabbit, guinea pig, mink or hamster. In one embodiment, the subject is treated with a therapeutic dose of the test agent for a period of at least 10 to 14 days. A sample, for instance, a brain sample, is obtained from the treated subject. In one embodiment, a brain sample includes brain tissue selected from the dorsal raphe nucleus (DRN) or the locus coeruleus (LC). The activity of the galaninergic system in the sample is then assayed. More particularly, the activity of the galaninergic system that is assayed is of a type correlated with antidepressant activity. Preferred activities include GalR2 mRNA or protein expression or ligand binding and galanin mRNA or protein expression. For assaying galanin mRNA expression, the galaninergic activity may be assayed by a quantitative amplification reaction, e.g., a quantitative reverse transcriptase-polymerase chain reaction (RT-PCR), using a galanin mRNA primer. For assaying galanin protein expression, an anti-galanin antibody or other galanin ligand, e.g., a galanin receptor such as a truncated GalR, may be employed. For assaying GalR2, the galaninergic activity may be assayed using a GalR2 protein or binding assay. The activity of the galaninergic system in the sample is compared with a control sample for upregulated or base galaninergic activity. Upregulation of galaninergic activity may be indicated by upregulation either of galanin mRNA or protein expression or of GalR2 expression (mRNA or protein) or binding. If the galaninergic activity of the treated sample is upregulated, the agent may also be characterized as having possible antidepressant activity. If the comparative galaninergic activity in the sample is not upregulated, the agent likely does not have antidepressant activity.

If an agent is identified as one having possible antidepressant activity, the specificity of the agent may be determined. The sample, or a corresponding sample, may be assayed for GalR1 expression or activity. The GalR1 expression or activity in the sample may then be compared to a control sample for upregulated or base GalR1 expression or activity. If GalR1 expression or activity in the sample is upregulated, the agent likely lacks specificity for antidepressant activity. If GalR1 expression or activity in the sample is not substantially altered, for instance, not substantially upregulated, e.g., P<0.05, the agent may have specificity for antidepressant activity.

### BRIEF DESCRIPTION OF THE FIGURES

FIG. 1 illustrates a series of photographs of immunofluorescent labeled serotoninergic neurons in the dorsal raphe nucleus (DRN) and the locus coeruleus (LC) showing galanin-like immunoreactivity in the rat DRN and LC. (A) Immunofluorescent double labeling of tryptophan hydroxylase (TPH, in green in color photograph) and galanin (GAL, in red in color photograph) in DRN. (B) Immunofluorescent double labeling of dopamine β-hydroxylase (DBH, in green in color photograph) and galanin (in red in color photograph) in the LC. (Scale bar, 50 μm).

FIG. 2 illustrates a series of bar graphs showing the effects of antidepressant treatments on galanin mRNA expression. Adult male rats were subjected to one of the three antidepressant treatments, i.e., 24 hour sleep deprivation, electroconvulsive shock (four shocks daily for 2 days), or FLX (10 mg/kg i.p. for 14 days). Different brain regions were dissected and tissues from 6-12 rats were pooled. Total RNAs were extracted and reverse transcribed with oligo(dT) primers. Real-time PCRs were repeated three times on pooled samples (A and B). (A) Galanin mRNA in different regions of naive rat brain was quantified and expressed as arbitrary unit. (B) Antidepressant treatments increased galanin mRNA expression in various brain regions. (C) FLX treatment increased galanin mRNA in the DRN and LC. Additional experiments were conducted on three independent pools of four rats/region/treatment to verify the effects of chronic FLX administration on galanin mRNA levels in the DRN and LC. *, P<0.05, Ctrl vs. FLX, Student's t test. PFC, prefrontal cortex; Amy, amygdala; Hip, hippocampus; PVN, paraventricular nucleus of hypothalamus; SD, sleep deprivation; ECS, electroconvulsive shock.

FIG. 3 illustrates two bar graphs showing how chronic FLX treatments up-regulate GalR2 protein levels in DRN. After electroconvulsive shock (four shocks per day for 2 days) or chronic (14 days) FLX (10 mg/kg, i.p.) treatment, the levels of GalRs in DRN and LC were determined with saturating [¹²⁵I]galanin binding (1 nM). Each treatment group included 12-18 rats, and samples from three animals were combined. Total galanin-binding sites (the sum of GalR1 and GalR2) and GalR2 sites were estimated by using 5 μM galanin (1-29) and 5 μM galanin (2-11) as competitors, respectively. Because GalR3 is not abundant in these regions, the difference between the total GalRs and GalR2 was defined as GalR1. The results are expressed as femtomoles per milligram protein ±SEM. Chronic FLX treatment increased GalR2 sites in DRN but not in LC (lower). None of the antidepressant treatments has effects on GalR1 sites (upper). *, P<0.05, Bonferroni t test for multiple comparisons with single control group.

FIG. 4 illustrates a first graph showing the displacement of [¹²⁵I]galanin binding by M40 from membranes prepared from GalR1- and GalR2-expressing cells and a second graph which is a bar graph showing results from rats pretreated for 14 days with FLX (10 mg/kg) or saline and given single i.c.v. infusion of the galanin receptor antagonist M40 or vehicle (ACSF) 45 minutes before testing in the forced swim test. Galanin receptor antagonist, M40, attenuated antidepressant-like effect of FLX in the forced swim test. (A) Displacement of [¹²⁵I]galanin binding by M40 from membranes prepared from GalR1- and GalR2-expressing cells. M40 has similar affinity for GalR1 and GalR2 with Kᵢ values of 1.8 nM and 5.1 nM, respectively. (B) Results from rats pretreated for 14 days with FLX (10 mg/kg) or saline and given single i.c.v. infusion of the galanin receptor antagonist M40 or vehicle (ACSF) 45 minutes before testing in the forced swim test. Activity was measured during a 10 minute test. Data represent group means (±SEM) of percentage time spent active in forced swim test. *, significance between saline/ACSF vs. FLX/ACSF. Rats pretreated for 14 days with FLX exhibited about a 46% increase in time spent active in the forced swim test compared to saline-pretreated rats; this effect was significant [P<0.05, Fisher's least significant difference (LSD) test]. This effect of FLX pretreatment was completely reversed by i.c.v. administration of M40. **, significance of FLX/ACSF vs. FLX/M40, P<0.01, Fisher's LSD.

FIG. 5 depicts graphs showing the results of the forced swim test with different doses of galnon and the results of the open field test with different doses of galnon. First graph: Activity was measured in the forced swim test during a 10 minute test at 45 minutes after injection. Higher doses of galnon produced a significant increase in activity in the test, compared to vehicle treated animals. As a positive control, one group was treated with the tricyclic antidepressant, desipramine (15 mg/kg, i.p.), which also displayed significantly increased activity. Second graph: Identical doses of drugs were administered in the open field test. Higher doses of galnon and desipramine significantly reduced activity in this task. (*, P<0.05 vs. control, Fisher's least significant difference test).

### DETAILED DESCRIPTION OF THE INVENTION

Selective serotonin reuptake inhibitors (SSRIs), such as FLX, are the most commonly used drugs in the treatment of major depression. However, there is a limited understanding of their molecular mechanism of action. While the acute effect of SSRIs in elevating synaptic serotonin concentrations is well known, the clinical amelioration of depressive symptoms requires 14-21 days of treatment, suggesting that numerous other rearrangements of function in the CNS must take place.

It is disclosed herein that 14 days of FLX treatment upregulated galanin mRNA levels by 100% and GalR2 binding sites by 50% in the rat dorsal raphe nucleus (DRN), where galanin coexists with serotonin. Furthermore, a galanin receptor antagonist, M40, attenuated the antidepressant like effect of FLX in the forced swim test, a rodent preclinical screen commonly used to evaluate antidepressant like efficacy. Direct activation of galanin receptors by a galanin receptor agonist, galnon, was found to produce an antidepressant like effect in the same task. Two other antidepressant treatments also affected the galaninergic system in the monoaminergic nuclei: electroconvulsive shock elevated galanin mRNA levels in DRN, while sleep deprivation increased galanin mRNA levels in the LC, further underlining the connection between activation of the galaninergic system and antidepressant action of various clinically proven treatments.

The invention thus provides a method for screening a drug candidate for probable antidepressant activity. In one embodiment, the method includes treating a subject with a therapeutic dose of the antidepressant drug candidate. In one embodiment, the subject is a nonhuman mammal, e.g., a rodent including a mouse, rat, hamster, rabbit, guinea pig, or mink. In one embodiment, brain tissue from the treated subject is dissected and the activity of the galaninergic system of a type correlated with antidepressant activity, e.g., selected from GalR2 expression and galanin mRNA expression, is assayed. The activity of the galaninergic system of the subject is compared to a control activity, and an upregulation or a lack of upregulation of galaninergic activity in the subject relative to the control is determined. If the comparative galaninergic activity of the subject is upregulated, the drug candidate is one which has possible antidepressant activity. If the comparative galaninergic activity of the subject is not upregulated, the drug candidate is one that does not have probable antidepressant activity.

In one embodiment, the subject is treated with a therapeutic dose of the antidepressant drug candidate for a period of at least 10 to 14 days. In one embodiment, the brain tissue is dissected, and a sample which includes certain regions of the brain are analyzed. In one embodiment, those regions are selected from the DRN and/or the LC. To quantitate galaninergic activity, any suitable method may be employed. For instance, to detect galanin expression, galanin RNA or protein may be detected. In one embodiment, galanin mRNA is detected in an amplification reaction, e.g., a quantitative PCR, which employs a galanin mRNA primer. In another embodiment, an antibody specific for galanin may be employed. Increased amounts or levels of galanin mRNA or protein (upregulation) is indicative of upregulation of galaninergic activity. In another embodiment, GalR2 RNA or protein expression or binding, is detected or determined. For instance, a GalR2 binding assay may be employed to detect or determine galaninergic activity. Increased amounts or levels of GalR2 binding or expression is indicative of an upregulation of galaninergic activity.

In one embodiment, the invention further includes detecting or determining whether the drug candidate has possible antidepressant activity. For example, the same or a corresponding brain sample as that employed to detect or determine galaninergic activity is assayed for GalR1 expression. In one embodiment, GalR1 binding is detected or determined. In another embodiment, the amount or level of GalR1 mRNA or protein expression is detected or determined. The GalR1 expression or binding in the sample from the subject is compared to a control for GalR1 expression or binding, and an upregulation or a lack of upregulation of GalR1 expression or binding in the subject relative to the control is determined. If the comparative GalR1 expression is upregulated, the drug candidate probably lacks specificity for antidepressant activity. If the comparative GalR1 expression is not upregulated, the drug candidate possibly has specificity for antidepressant activity.

The invention will be further described by the following nonlimiting example.

### EXAMPLE

Material and Methods

Animals. Adult male Sprague Dawley rats (Harlan, Indianapolis, Ind.), weighing 250 275 grams, were given ad libitum access to food and water and were maintained on a 12 hour light/dark cycle. All procedures were conducted in accordance with the National Institutes of Health Guide for the Care and Use of Laboratory Animals.

Sleep deprivation and electroconvulsive shock. Twenty-four hour sleep deprivation was achieved by disturbing the rats whenever sleep behavior was observed. For electroconvulsive shock treatment, rats received four shocks bilaterally each day, delivered using a constant current UGO Basile apparatus for small mammals (Varese, Italy; 90 mA, 70 Hz, until a tonic clonic seizure developed), separated by 1 hour interval, for 2 days.

Immunohistochemistry. Adult male rats were deeply anesthetized, perfused with 4% paraformaldehyde and 30 μm coronal sections were cut with a cryostat. Sections were permeablized in 0.1% Triton X 100 for 30 minutes, blocked with 10% normal goat serum for 1 hour, and incubated with the following primary antibodies: rabbit polyclonal galanin (Bachem Bioscience Inc., King of Prussia, Pa.; 1:5000) and mouse monoclonal trypotophan hydroxylase (Sigma, Saint Louis, Mo.; 1:1000) overnight at room temperature. Goat anti rabbit Alexa Fluor 594 and goat anti mouse Alexa Fluor 488 (Molecular Probe, Eugene, Oreg.) were used at 1:400 for 2 hours at room temperature. The sections were examined with a confocal scanning microscope (Olympus Optical, Tokyo, Japan) equipped with the appropriate filter combinations.

Tissue dissections. Tissues used for RNA extraction or binding study were rapidly dissected after sacrifice and immediately frozen on dry ice. All dissections were performed by an experienced neuroanatomist with a rat brain slicer and for small structures, like LC and DRN, the punching method was used to assure reproducible dissection.

Quantitative real time PCR. Brain tissues obtained from 12 rats (DRN, LC, paraventricular nucleus of hypothalamus) or 6 rats (prefrontal cortex, amygadala, hippocampus) were pooled and total RNA was prepared with Trizol reagent (Invitrogen, Carlsbad, Calif.) following manufacturer's instructions. For galanin mRNA quantification in DRN and LC after FLX treatment, the experiment was repeated with 3 independent pools of 4 rats per treatment per brain region. Aliquots of total RNA (2 μg) and oligo(dT) primers were then reverse transcribed with ThermoScript RT (Invitrogen) at 51° C. for 60 minutes. Quantitative PCR was performed using a Roche LightCycler and LightCycler Fast Start DNA Master SYBR Green I mix (Roche Applied Science, Indianapolis, Ind.). Specific primers (β-actin: 5′ GGC TAC AGC TTC ACC ACC AC 3′ (SEQ ID NO:1) and 5′ TGC GCT CAG GAG GAG C 3′ (SEQ ID NO:2); galanin: 5′ AGG CAA GAG GGA GTT ACC ACT 3′ (SEQ ID NO:3) and 5′ GGT GGC CAA GGG GAT G 3′ (SEQ ID NO:4)) were designed to correspond to sequences on different exons to avoid amplification of possible genomic DNA contamination. Real time PCR assays included an initial 10 minutes, 94° C. step to activate Taq polymerase, followed by 35 cycles of denaturation 94° C., 10 seconds, annealing 58° C., 10 seconds, and extension 72° C., 25 seconds. The results were expressed in arbitrary units normalized by the expression levels of the reference gene, β-actin.

Cell lines. Stably transfected Chinese Hamster Ovary (CHO) cells expressing rat GalR2, and Bowes' melanoma cells that express human GalR1, were cultivated as described earlier (Wang, S., et al. Biochemistry 1998, 37, 6711-7; and Heuillet, E., et al. Eur. J. Pharmacol. 1994, 269, 139-47).

Membrane preparation and binding assay. DRN and LC were dissected from a total of 16 rats per group and samples from 4 animals were pooled. Rat brain synaptic membranes (Lu, X., et al. Neurosci. Lett. 2000, 286, 149-53) and cells were prepared as previously described (Heuillet, E., et al. Eur. J. Pharmacol. 1994, 269, 139 47). Equilibrium binding of ¹²⁵I, galanin (2200 Ci/mmol, PerkinElmer Life Science, Boston, Mass.) to hippocampal membrane preparations was performed in 150 μL binding buffer [50 mM Tris Cl (pH 7.4), 5 mM MgCl2, 0.05 % (w/v) bovine serum albumin, supplemented with protease inhibitors]. Incubations were at room temperature for 45 minutes and terminated by rapid filtration through glass fiber filters (Packard, Meriden, Conn.). After three washes with cold PBS (pH 7.4) containing 0.01 % (v/v) Triton X 100, the filter was counted with Cobra II auto Gamma counting systems (Parkard Bioscience, Downer Grove, Ill.).

To determine the sum of GalR1 and GalR2 receptor sites in tissues, ¹²⁵I galanin was used at a saturating concentration of 1 nM and nonspecific binding was determined in the presence of 5 μM of galanin (Duman, R. S., et al. Arch. Gen. Psychiatry 1997, 54, 597-606; Mann, J. J. Neuropsychopharmacology 1999, 21, 99S-105S; Schloss, P., et al. Pharmacol. Ther. 2004, 102, 47-60; Branchek, T. A., et al. Trends Pharmacol. Sci. 2000,21, 109-17; Vrontakis, M. E. Curr. Drug Targets CNS Neurol. Disord. 2002, 1, 531 41; Wrenn, C. C., et al. Prog. Neuropsychopharmacol. Biol. Psychiatry 2001, 25, 283-99; Xu, Z. Q., et al. Neuroscience 1998, 87, 79-94; Weiss, J. M., et al. Ann. N.Y. Acad. Sci. 1998, 863, 364-82; Melander, T., et al. Journal of Neuroscience 1986, 6, 3640-54; Skofitsch, G., et al. Peptides 1985, 6, 50946; O'Donnell, D., et al. J. Comp. Neurol. 1999, 409, 469-81; Priestley, J. V., et al. J. Neurosci. Methods 1993, 48, 99-110; Gallager, D. W., et al. Neuropharmacology 1976, 15, 149-56; Aghajanian, G. K., et al. Brain Res. 1978, 153, 169-75; Zachariou, V., et al. Journal of Neurochemistry 2001, 76, 191-200; Zachariou, V., et al. Proc. Natl. Acad. Sci. U.S.A. 2003, 100, 9028-33; Barr, A. M., et al. Trends Pharmacol. Sci. 2002, 23, 475-82; Markou, A., et al. Neuropsychopharmacology 1991, 4, 17-26; Bellido, I., et al. Neuroscience Letters 2002, 317, 101-5; Husum, H., et al. Neuropsychopharmacology 2003, 28, 1292-9; Sergeyev, V., et al. Psychopharmacology 2004, in press; Murck, H., et al. Psychoneuroendocrinology 2004, 29, 1205-11; Yoshitake, T., et al. Neuropharmacology 2003, 44, 206-13; Yoshitake, T., et al. Proc. Natl. Acad. Sci. U.S.A. 2004, 101, 354-9; Bartfai, T., et al. Proc. Natl. Acad. Sci. U.S.A. 2004, 101, 10470-5; Szuba, M. P., et al. Depress. Anxiety 2000, 12, 170-7; Nolen, W. A., et al. Int. Clin. Psychopharmacol. 1989, 4, 217-28; Zhao, Y., et al. J. Pharmacol. Exp. Ther. 2003, 307, 246-53; and Heal, D. J., et al. J. Neurochem. 1989, 53, 1019-25). The number of GalR2 sites was estimated by using 5 μM of the highly selective GalR2 ligand, galanin(2-11), as a competitor (Mann, J. J. Neuropsychopharmacology 1999, 21, 99S-105S; Schloss, P., et al. Pharmacol. Ther. 2004, 102, 47-60; Branchek, T. A., et al. Trends Pharmacol. Sci. 2000, 21, 109-17; Vrontakis, M. E. Curr. Drug Targets CNS Neurol. Disord. 2002, 1, 531-41; Wrenn, C. C., et al. Prog. Neuropsychopharmacol. Biol. Psychiatry 2001, 25, 283-99; Xu, Z. Q., et al. Neuroscience 1998, 87, 79-94; Weiss, J. M., et al. Ann. N.Y. Acad. Sci. 1998, 863, 364-82; Melander, T., et al. Journal of Neuroscience 1986, 6, 3640-54; Skofitsch, G., et al. Peptides 1985, 6, 50946; and O'Donnell, D., et al. J. Comp. Neurol. 1999, 409, 469-81) (Liu, H. X., et al. Proceedings of the National Academy of Sciences of the United States of America 2001, 98, 9960-4). As GalR3 is not abundant in the regions tested, GalR1 sites were estimated by subtracting GalR2 sites from the sum of GalR1 and GalR2 receptor sites. The selectivity of galanin (Mann, J. J. Neuropsychopharmacology 1999, 21, 99S-105S; Schloss, P., et al. Pharmacol. Ther. 2004, 102, 47-60; Branchek, T. A., et al. Trends Pharmacol. Sci. 2000, 21, 109-17; Vrontakis, M. E. Curr. Drug Targets CNS Neurol. Disord. 2002, 1, 531-41; Wrenn, C. C., et al. Prog. Neuropsychopharmacol. Biol. Psychiatry 2001, 25, 283-99; Xu, Z. Q., et al. Neuroscience 1998, 87, 79-94; Weiss, J. M., et al. Ann. N.Y Acad. Sci. 1998, 863, 364-82; Melander, T., et al. Journal of Neuroscience 1986, 6, 3640-54; Skofitsch, G., et al. Peptides 1985, 6, 50946; and O'Donnell, D., et al. J. Comp. Neurol. 1999, 409, 469-81) for the GalR2 receptors over GalR1 receptors in the applied concentration has been confirmed by binding assays with GalR1 and GalR2 expressing cells (Mazarati, A., et al. Eur. J. Neurosci. 2004, 19, 3235 44).

Surgery. Rats underwent aseptic stereotaxic surgery under ketamine (100 mg/kg, i.p.) xylazine (10 mg/kg, i.p.) anesthesia. A guide cannula 1.4 cm in length, of 26 gauge stainless steel hypodermic tubing (Plastics One, St. Louis, Mo.) was implanted into the right lateral ventricle at coordinates 0.5 mm posterior, 1.0 mm lateral to bregma, and 3.5 mm ventral to the surface of the skull. A 31 gauge stylet was secured to the guide cannula following the surgery. After surgery, rats were given at least 8 days recovery before the start of behavioral testing.

Drug treatments. Fluoxetine (FLX, Sigma, St. Louis, Mo.) was dissolved in saline and administered i.p. at a dose of 10 mg/kg daily for 14 days for biochemical studies. Separate groups of rats received daily i.p. injections of FLX or saline vehicle for 14 days for testing in the forced swim task. On day 14, rats were administered FLX or saline i.p. at 45 minutes prior to the forced swim test and M40 (Bachem, King of Prussia, Pa.) or ACSF i.c.v. at 15 minutes prior to the forced swim test (see methods below). Single i.c.v. microinjections of M40 (8 nmol/2 μl ACSF) or ACSF (2 μl) were performed with a 10 μl Hamilton syringe connected via Becton Dickinson polyethylene tubing (PE20) to a 1.5 cm injector (Plastics One) fabricated from 31 gauge hypodermic tubing. The animal was allowed to freely explore a small cage during the infusion of 2 μl over 1.5 minutes, with each 1 μl separated by ten seconds, and an additional sixty seconds before the injector was withdrawn from the guide cannula. The treatments consisted of equal numbers of rats receiving one of the following combinations of treatments: saline+ACSF, saline+M40, FLX+ACSF, or FLX+M40. Additional treatment groups (n=10 per group) were examined in a separate forced swim test 45 minutes after i.p. injection of one of the following: 50% DMSO (w/v), galnon (Vulpes, Estonia; dissolved in 50% DMSO), or desipramine (Sigma, St Louis, Mo., 15 mg/kg in 50% DMSO). One week after the forced swim test, rats were randomly assigned to treatment groups. Rats were treated with vehicle, galnon or desipramine. Forty five minutes after injection, rats were subjected to the open field test.

Forced swim test. Rats were placed singly in a cylindrical glass container (48 cm height, 21 cm diameter) with tap water (25° C.) to a depth of 27 cm and tested for 10 minutes. Water was changed and the cylinder thoroughly rinsed after each test. Tests were recorded on videotape and later scored by an experienced observer blind to the experimental conditions of the rats. Behaviors were scored as either (Duman, R. S., et al. Arch. Gen. Psychiatry 1997, 54, 597-606) active B characterized as when the rat engages in escape behaviors, typically evident by forepaws splashing above the surface of the water, (Mann, J. J. Neuropsychopharmacology 1999, 21, 99S-105S) inactive B described by minimal locomotor activity. The presence of antidepressant activity in a drug is inferred from its capacity to increase the fraction of time spent in active behavior (Galea, L. A., et al. Behav. Brain Res. 2001, 122, 1-9).

Open field test. One week after the forced swim test, rats were randomly assigned to treatment groups. Rats were treated with vehicle, galnon or desipramine; 45 minutes after injection, rats were placed in an open field. The apparatus consisted of a square arena (100 by 100 cm), with a 40 cm high opaque white wall. The floor was marked into 9 equal segments and fluorescent light provided diffuse overhead illumination. Locomotor activity over 5 minutes was recorded on videotape. Tapes were later scored blind to the experimental condition of the animals. Line crossing behavior (defined as at least three paws in a square) was tallied and compared between experimental conditions.

Statistical methods. Data for forced swim test studies utilizing galnon and desipramine were subject to a one factor analysis of variance (ANOVA), with drug treatment as the between subjects factor. For the M40 FLX study, data were subject to a two factor between subjects design, with chronic drug treatment (FLX vs vehicle) and i.c.v. drug (M40 vs ACSF) as the main factors. Significant main effects were followed up with Fisher's LSD posthoc test to determine specific group differences. Data for the GalR1 and GalR2 binding sites in DRN and LC following FLX treatment were analyzed by the Student t-Test with the Bonferroni correction applied to control alpha level for multiple comparisons with single control group.

Results

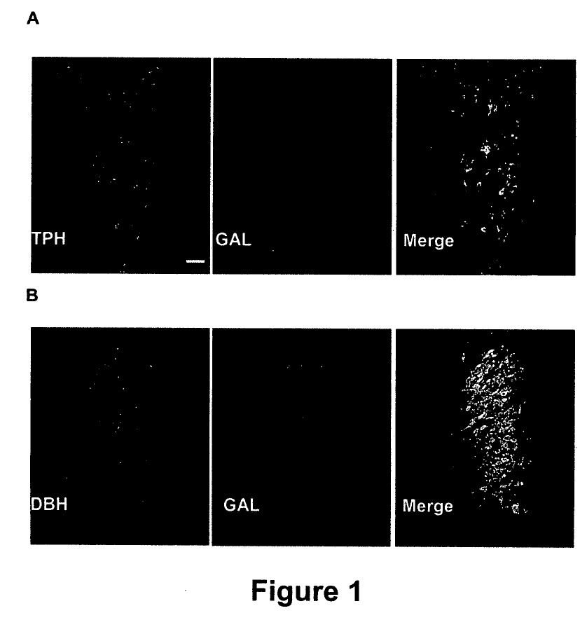

Galanin like immunoreactivity (Galanin LI) is abundant in serotoninergic and noradrenergic nuclei. Previous immunohistochemical studies on colchicine treated animals have shown that galanin is widely distributed in the central nervous system, and galanin-LI usually colocalizes with cholinergic, catecholaminergic and serotoninergic markers (Melander, T., et al. Journal of Neuroscience 1986, 6, 3640 54; and Skofitsch, G., et al. Peptides 1985, 6, 509-46). Double immunofluorescent labeling techniques were employed to reexamine the distribution of galanin-LI in the noradrenergic and serotoninergic systems in non colchicine treated naive rats. In the DRN, as expected from the labeling of serotoninergic neurons, tryptophan hydroxylase immunoreactivity was mainly present in cell bodies and primary dendrites. Most tryptophan hydroxylase positive neurons exhibited moderate galanin-LI, whereas non serotoninergic galanin-LI fibers and cell bodies were also present (FIG. 1A). In the LC, strong galanin-LI was observed in both cell bodies and fibers, showing almost complete colocalization with the noradrenergic marker, dopamine β-hydroxylase (FIG. 1B). The merged images demonstrated that almost all the noradrenergic neurons in the LC were galanin positive.

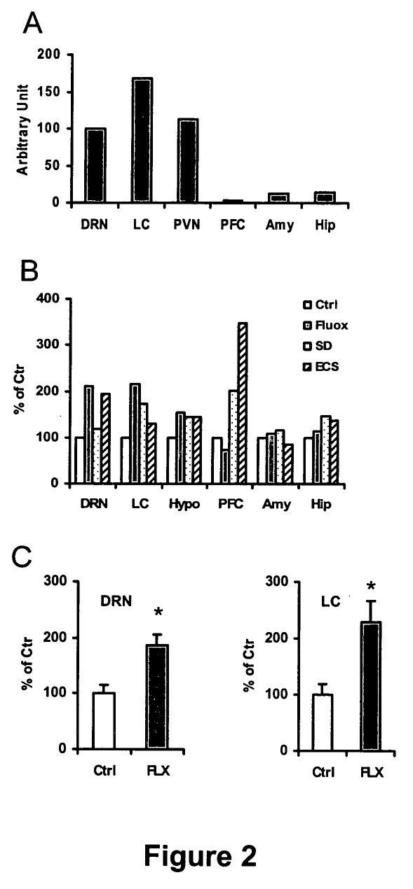

Quantification of galanin mRNA in the rat brain. Galanin mRNA expression levels, among the six brain regions tested, were highest in paraventricular nucleus of hypothalamus, LC and DRN (FIG. 2A). High expression levels of galanin mRNA in hypothalamus, LC and DRN have previously been suggested by Northern blot analysis and in situ hybridization (Gundlach, A. L., et al. Neurosci. Lett. 1990, 114, 241-7; and Kaplan, L. M., et al. Proc. Natl. Acad. Sci. U.S.A. 1988, 85, 1065-9). Amygdala and hippocampus have similar levels of galanin mRNA, both representing approximately 13% of that of DRN. Galanin mRNA expression was also detected in the prefrontal cortex, with roughly 3% of the DRN level.

Antidepressant treatments upregulate galanin mRNA expression in several brain regions. The effects of three clinically relevant antidepressant treatments, i.e., chronic FLX i.p. injections for 14 days, 24 hour sleep deprivation, or electroconvulsive shock for 2 days (4 shocks daily with 1 hour interval), on galanin mRNA expression were analyzed using real time PCR (FIG. 2B). In prefrontal cortex, DRN and LC, two out of three antidepressant treatments produced a marked increase in galanin mRNA levels. In the DRN, the electroconvulsive shock and chronic FLX treatment had similar effects, resulting in a 2-fold increase; in the LC, FLX and sleep deprivation resulted in a 2.2- and 1.8-fold increase, respectively. Additional studies were conducted to better quantify the effects of chronic FLX administration on galanin mRNA levels in DRN and LC on separate pools of tissue samples from animals treated with saline and FLX. The results (FIG. 2C) were similar to those obtained from larger pools of samples (FIG. 2B).

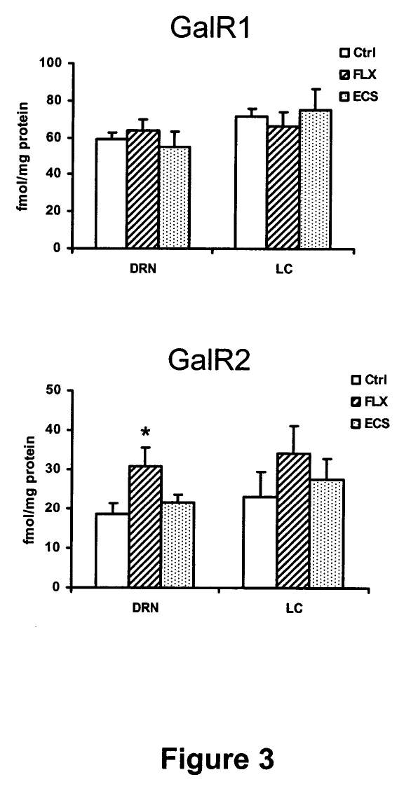

Chronic FLX treatment increases GalR2 binding sites in DRN. In control animals, both LC and DRN showed high levels of galanin binding sites (FIG. 3), with GalR1 sites estimated at 58 fmoles/mg protein in DRN and 72 fmoles/mg protein in LC. Approximately one third of total galanin binding sites corresponded to GalR2 receptors (33% in DRN and 32% in LC), a finding that is consistent with previous observations on GalR1 and GalR2 mRNA distribution obtained using in situ hybridization (O'Donnell, D., et al. J. Comp. Neurol. 1999, 409, 469 81; and Gustafson, E. L., et al. Neuroreport. 1996, 7, 953-7).

In the DRN, FLX treatment significantly increased the number of GalR2 sites by 58%, without changes in the number of GalR1 binding sites (FIG. 3). Electroconvulsive shock had no effect on either GalR1 or GalR2 sites. In the prefrontal cortex, hippocampus, and amygdala, none of the antidepressant treatments led to significant changes in the levels of either GalR1 or GalR2 receptors (data not shown).

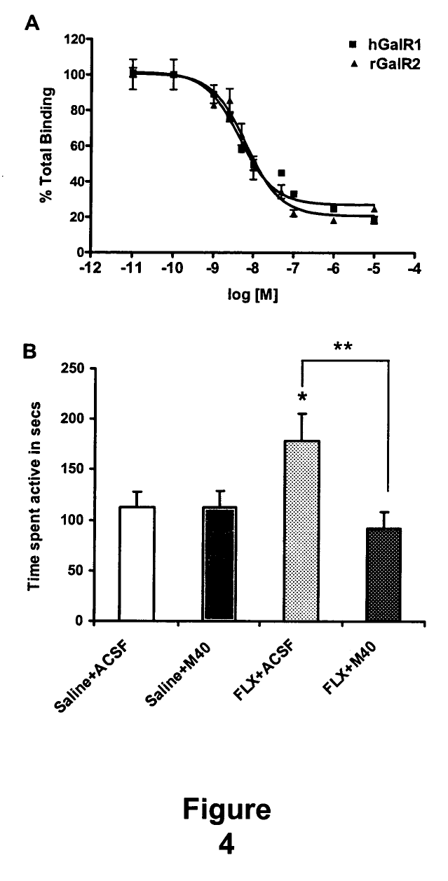

Galanin receptor antagonist, M40. attenuated antidepressant like effect of FLX in the forced swim test. The Ki of M40 for GalR1 and GalR2 receptors were 1.82 nM and 5.1 nM, respectively (FIG. 4A). The data on the effects of FLX and M40 in the forced swim test (FIG. 4B) was subjected to a two factor ANOVA. Results indicated a significant main effect of treatment [F_{(1,40)}=5.568 p<0.05] and a significant interaction between pretreatment (FLX or saline) and treatment (M40 or ACSF) [F_{(1,40)}=5.520 p<0.05]. Subsequent post hoc analysis showed that FLX, when administered at 10 mg/kg (i.p. 14 days), produced a significant increase in the time that rats spent active in the forced swim test, suggestive of an antidepressant like effect (p<0.05, FLX+ACSF vs Saline+ACSF). The galanin receptor antagonist M40, when infused 15 minutes prior to the forced swim test, at a dose shown to block galanin induced increase in food intake (8.0 nmol, i.c.v), significantly attenuated the increased time spent active in the forced swim test, thus antagonizing the putative antidepressant like effect of FLX (p<0.01, FLX+ACSF vs FLX+M40) (FIG. 4B).

Galnon, a galanin receptor agonist, shows behavioral effects in forced swim test and open field test. The systemically active, nonpeptide galanin receptor agonist, galnon, exhibited moderate affinity for both GalR1 (Saar, K., et al. Proc. Natl. Acad. Sci. U.S.A. 2002, 99, 7136-41) and GalR2. It was observed that galnon displaces ¹²⁵I-galanin from both GalR1 and GalR2 receptors with Ki of 11.7 μM and 34.1 μM, respectively (data not shown).

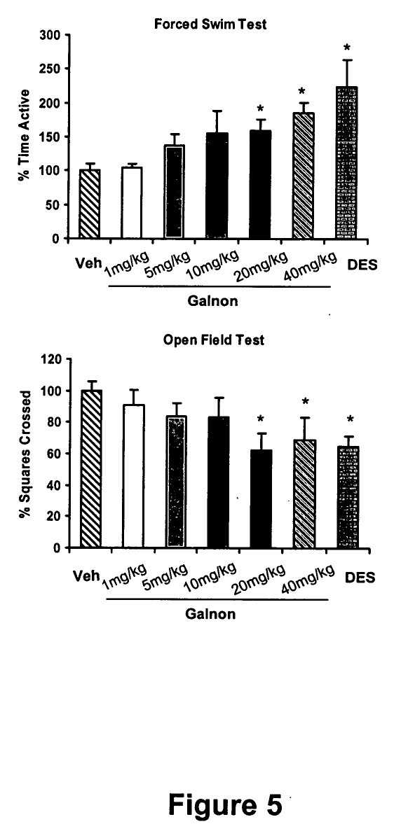

Galnon, in doses ranging from 1-40 mg/kg i.p., was tested in the forced swim test with desipramine (15 mg/kg, i.p.) as a positive control (FIG. 5). The data from the forced swim test following galnon administration were subjected to a one factor analysis of variance (ANOVA), with drug treatment as the factor. Results of the ANOVA indicated a significant main effect of drug treatment [F_{(7,61)}=3.98, p<0.001]. Further post hoc analysis revealed that galnon produced a dose dependent increase in activity in the forced swim test. The effects were significant at 20 mg/kg (p<0.05) and 40 mg/kg doses (p<0.01). Desipramine, used as a positive control, produced a robust increase in activity (125%, p<0.001).

Data from the open field test (FIG. 5) with galnon and desipramine were subjected to a one factor ANOVA, with drug treatment as the factor. The results of the ANOVA revealed a significant main effect of drug treatment [F_{(7,61)}=3.35, p<0.01]. Post hoc analysis indicated that increasing doses of galnon tended to decrease activity in the open field, as activity was significantly reduced in the two higher doses of 20 mg/kg (p<0.01) and 40 mg/kg (p<0.05) galnon. Desipramine also produced a significant reduction (p<0.01) of locomotor activity, an effect that has been consistently described in the literature (Mague, S. D., et al. J. Pharmacol. Exp. Ther. 2003, 305, 323-30).

Discussion

Previous reports are confirmed herein that the DRN and LC express high levels of galanin-LI (Melander, T., et al. Journal of Neuroscience 1986, 6, 3640-54; and Skofitsch, G., et al. Peptides 1985, 6, 509-46) and that galanin-LI is colocalized partially with tryptophan hydroxylase immunoreactivity in the DRN and almost completely with dopamine β-hydroxylase immunoreactivity in the LC (FIG. 1). A 100% increase in galanin mRNA in the DRN was observed following two antidepressant treatments: electroconvulsive shock for 2 days and FLX treatment for 14 days (FIG. 2). A similar large increase in galanin mRNA in the LC was induced after treatment with FLX (14 days) and sleep deprivation (24 hours). Previous studies have demonstrated that increase in galanin mRNA expression is associated with an increase in galanin synthesis and release (Meister, B., et al. Cell Tissue Res. 1990, 260, 279-97; Villar, M. J., et al. Brain Res. 1994, 650, 219-28; and Landry, M., et al. Neuroscience 1998, 84, 897-912). The finding that three different antidepressant treatments strongly elevated galanin mRNA levels at time points when clinically relevant antidepressant effect become effective suggests that their antidepressant like activity might be associated with induction of galanin synthesis and release.

Concurrent with the increase in galanin peptide expression, an increase in GalR2 binding sites following FLX treatment was detected (FIG. 3B). Simultaneous increases in galanin expression and GalR2 binding may indicate that the GalR2 is not a readily desensitizing receptor subtype. GalR1 receptor sites in DRN were unaltered by chronic FLX treatment and remained at pretreatment levels, whereas GalR2 receptor binding sites were elevated by 50%, resulting in a relative shift in the effects of galanin on DRN neurons towards a greater influence exerted through GalR2. As GalR1 acts through the Gᵢ mediated inhibition of adenylyl cyclase, whereas GalR2 acts through Gq/G₁₁ mediated increase in IP3 and intracellular Ca²⁺ concentrations (Wang, S., et al. Biochemistry 1998, 37, 6711-7), it is disclosed herein that the FLX treatment induced “bias” towards increased GalR2 mediated influence on DRN neurons would result in increased firing rates in these neurons. Indeed, excitatory effects of galanin, exerted probably through GalR2, on neurotransmitter release have been reported in some brain regions (Ogren, S. O., et al. Neurosci. Lett. 1991, 128, 253-6; and Ogren, S. O., et al. Ann. N.Y Acad. Sci. 1998, 863, 342-63). Furthermore, activation of dorsal hippocampal GalR2 receptors facilitates cognition while activation of GalR1 in the ventral hippocampus impairs cognitive performance (Ogren, S. O., et al. Ann. N.Y. Acad. Sci. 1998, 863, 342-63), suggesting that a change in the overall action of galanin would occur when the balance between GalR1 and GalR2 is altered.

The relevance of the above findings, which links increases in galanin mRNA and GalR2 receptors in the DRN to the antidepressant like effect of FLX, was underscored by the findings that the galanin receptor antagonist, M40 (Bartfai, T., et al. Proc. Natl. Acad. Sci. U.S.A. 1993, 90, 11287-91), attenuated the antidepressant like effect of FLX in the forced swim test (FIG. 4B). These data suggest that the increased galanin mRNA and GalR2 are relevant for the antidepressant like effect of this widely used antidepressant drug.

Galnon, the low molecular weight, galanin receptor agonist, displayed antidepressant like effect in the forced swim test (FIG. 5). Galnon, in similar doses has been shown to inhibit signs of opiate withdrawal (Zachariou, V., et al. Proc. Natl. Acad. Sci. U.S.A. 2003, 100, 9028-33), and to increase the latency of phenyleneterazol induced seizures (Saar, K., et al. Proc. Natl. Acad. Sci. U.S.A. 2002, 99, 7136-41). Galnon is a non selective agonist for GalR1/GalR2, thus its acute antidepressant like effect is probably similar to the effects of increased galanin releaseBenhanced by antidepressant treatment. In the acute forced swim test, GalR2 levels are unlikely to be adjusted as they are after 14 days treatment with FLX due to the short time available between injection and testing, and thus, a change in GalR1/GalR2 ratio in the DRN is not likely to explain the antidepressant like effect of galnon. Other aspects of galanin actions might contribute to the acute effect, for instance, through a direct postsynaptic effect on the prefrontal area, where galanin mRNA levels were also elevated following antidepressant treatments (FIG. 2B). A synergistic effect between galanin and 5-HT1A receptor activation has been reported (Hedlund, P. B., et al. Eur. J. Pharmacol. 1991, 204, 87-95). In addition, the effects of galanin in hypothalamus may contribute to antidepressant like effect as galanin regulates rapid eye movement (REM) sleep (Murck, H., et al. Psychoneuroendocrinology 2004, 29, 1205-11; Toppila, J., et al. Neurosci. Lett. 1995, 183, 171-4; and Murck, H., et al. J. Psychiatr. Res. 1999, 33, 225-32), feeding (Bartfai, T., et al. Critical Reviews in Neurobiology 1993, 7, 229-74), and hormone release (Bartfai, T., et al. Critical Reviews in Neurobiology 1993, 7, 229-74), all of which are disturbed in depression (Barr, A. M., et al. Trends Pharmacol. Sci. 2002, 23, 475-82). It has been reported that galanin exerts antidepressant like effect through suppression of REM sleep (Murck, H., et al. Psychoneuroendocrinology 2004, 29, 1205-11; Toppila, J., et al. Neurosci. Lett. 1995, 183, 171-4; Murck, H., et al. J. Psychiatr. Res. 1999, 33, 225-32 ; and Fujihara, H., et al. Brain Res. Mol. Brain Res. 2003, 119, 152-9). Other hypothalamically abundant neuropeptides such as corticotrophin releasing factor (Clark, M. S., et al. Semin. Clin. Neuropsychiatry 2003, 8, 119 36) and arginine vasopressin (van Broekhoven, F., et al. Psychopharmacology (Berl.) 2003, 165, 97-110) have also been implicated in mood regulation. Double immunolabeling studies have showed that while the coexistence between corticotrophin releasing factor and galanin is very low (Ceccatelli, S., et al. Neuroendocrinology 1989, 49, 309-23), the coexistence between arginine vasopressin and galanin is very common in the paraventricular nucleus (Gai, W. P., et al. J. Comp. Neurol. 1990, 298, 265-80) and it has been shown that the arginine vasopressin release can be regulated by galanin (Ciosek, J., et al. J. Physiol. Pharmacol. 2003, 54, 625-41). On the other hand, a recent study showed that REM sleep deprivation selectively up regulates galanin expression without affecting the mRNA levels of either corticotrophin releasing factor or arginine vasopressin (Fujihara, H., et al. Brain Res. Mol. Brain Res. 2003, 119, 152-9), suggesting that a galaninergic antidepressant like effect might be achieved without the influence of these additional neuropeptide systems.

The three antidepressant treatments disclosed herein, similar to the numerous studies on the mechanisms of action of antidepressant treatments, were carried out on naive, “non depressed” animals. Despite the differences in the neurochemistry of “depressed” and “naive” brain, antidepressant drug screening in naive animals has been proven useful (Cryan, J. F., et al. Trends Pharmacol. Sci. 2002, 23, 23845). In agreement with the disclosure herein with naive animals, an antidepressant effect of i.v. applied galanin in depressed patients was found (Murck, H., et al. Psychoneuroendocrinology 2004, 29, 1205-11).

### SUMMARY

The relevance of the galanin system for the treatment of depression was determined by examining the effects of three clinically validated antidepressant treatments, sleep deprivation (24 hours), electroconvulsive shock (4 shocks over 2 days) and, the most commonly used, chronic FLX treatment (14 days), on the expression levels of galanin and its receptors in the DRN and LC of the rat. The length of each treatment was chosen to correlate with the onset of clinical benefit of each treatment and previous experience in the animal studies (Szuba, M. P., et al. Depress. Anxiety 2000, 12, 170 7; Nolen, W. A., et al. Int. Clin. Psychopharmacol. 1989, 4, 217 28; Zhao, Y., et al. J. Pharmacol. Exp. Ther. 2003, 307, 246 53; and Heal, D. J., et al. J. Neurochem. 1989, 53, 1019 25). To further examine the contribution of altered galaninergic signaling to the FLX mediated antidepressant like effect, it was tested whether a galanin receptor antagonist, M40, blocked the antidepressant like effect of chronic FLX treatment (10 mg/kg i.p., 14 days) and whether a galanin receptor agonist, galnon, exerted an antidepressant like effect in the rat forced swim test. The results showed that M40 blocked the antidepressant like effect of chronic FLX treatment and galnon exerted an antidepressant like effect in the rat forced swim test.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method for detecting or determining one or more agents that alter GalR2 expression or activity or galanin expression, comprising:
a) providing a brain sample from a mammal administered an antidepressant amount of one or more test agents; and
b) detecting or determining whether GalR2 expression or activity or galanin expression in the sample is altered.

2. The method of claim 1 further comprising detecting or determining whether GalR1 expression or activity in the sample or a corresponding sample is altered.

3. The method of claim 1 wherein the mammal is not a human.

4. The method of claim 1 wherein the mammal is a rodent.

5. The method of claim 1 wherein GalR2 expression or activity in the dorsal raphe nucleus is detected or determined.

6. The method of claim 1 wherein GalR2 expression or activity in the locus coeruleus is detected or determined.

7. The method of claim 1 wherein GalR2 binding is detected or determined.

8. The method of claim 1 wherein galanin expression in the dorsal raphe nucleus is detected or determined.

9. The method of claim 1 wherein galanin expression in the locus coeruleus is detected or determined.

10. The method of claim 1 wherein the amount or level of galanin RNA is detected or determined.

11. The method of claim 2 wherein GalR1 expression or activity in the dorsal raphe nucleus is detected or determined.

12. The method of claim 2 wherein GalR1 binding is detected or determined.

13. A method for identifying an agent that has galaninergic activity associated with antidepressant activity, comprising:
a) comparing the amount or level of GalR2 expression or activity or galanin expression and the amount or level of GalR1 expression or activity in a test sample to the amount or level of GalR2 expression or activity or galanin expression and the amount or level of GalR1 expression or activity in a control sample, wherein the test sample is a brain sample from a mammal administered a test agent in an antidepressant amount; and
b) identifying an agent that increases GalR2 expression or activity or increased galanin expression without substantially altering GalR1 expression or activity.

14. The method of claim 13 wherein the mammal is not a human.

15. The method of claim 13 wherein the mammal is a rodent.

16. The method of claim 13 wherein the amount or level of GalR2 expression or activity in the dorsal raphe nucleus is compared.

17. The method of claim 13 wherein the amount or level of GalR2 expression or activity in the locus coeruleus is compared.

18. The method of claim 13 wherein the amount or level of galanin expression in the dorsal raphe nucleus is compared.

19. The method of claim 13 wherein the amount or level of galanin expression in the locus coeruleus is compared.

20. The method of claim 13 wherein the amount or level of GalR1 expression or activity in the dorsal raphe nucleus is compared.

21. A method for identifying one or more agents that alter GalR2 activity, comprising:
a) contacting one or more agents with a mammalian host cell comprising an expression cassette comprising a GalR2 open reading frame operably linked to a promoter; and
b) detecting or determining whether the one or more agents alter GalR2 expression or activity.

22. The method of claim 21 wherein the host cell is a human cell.

23. The method of claim 22 wherein the cell is a Hep293 cell.

24. The method of claim 21 wherein phosphoinositol-3 turnover is detected or determined.

25. The method of claim 21 wherein the expression cassette comprises a human GalR2 open reading frame.
